(19) United States
(12) Patent Application Publication
(10) Pub. No.: US 2006/0029789 A1
(43) Pub. Date: Feb. 9, 2006

(54) Interior vehicle trim panel having dual density spray elastomer and method for making the same

(75) Inventors: Joseph T. Donatti, Howell, MI (US); Nelson E. Williams JR., Saline, MI (US); Glenn A. Cowelchuk, Chesterfield, MI (US)

Correspondence Address: BROOKS KUSHMAN P.C. / LEAR CORPORATION, 1000 TOWN CENTER, TWENTY-SECOND FLOOR, SOUTHFIELD, MI, 48075-1238, US

(73) Assignee: Lear Corporation, Southfield, MI (US)

(21) Appl. No.: 10/912,617

(22) Filed: Aug. 5, 2004

(51) Int. Cl. A61F 5/00; B32B 3/26

(52) U.S. Cl. 428/304.4

(57) ABSTRACT

The present invention provides a vehicle interior trim panel. The panel may be made by providing a spray mold having a mold surface having a cavity having a portion having a height to depth (H/D) ratio of less than 1.0. Polyurethane material can be sprayed proximate the surface of the tool to form a polyurethane skin layer. Expandable polyurethane material can be sprayed onto the skin layer to form a resilient layer. The skin layer and the resilient layer can be removed from the tool and positioning in a mold in a spaced apart relationship from a substrate, with a foam material being introduced between the substrate and the resilient layer to form a foam layer therebetween.

### BACKGROUND OF THE INVENTION

1. Field of the Invention

The invention relates to interior vehicle trim panels having dual density spray elastomer and method for making the same.

2. Background Art

The use of interior trim panels in automotive applications is relatively well known. One relatively typical vehicle interior trim panel comprises a relatively rigid substrate having a flexible thin plastic skin disposed over at least an interior facing portion of a surface of the substrate. Spraying material, such as a polyurethane composition, onto a mold provides a skin having several advantages. When a softer touch for the panel is desired, foam has been provided between the substrate and the skin.

A common method of providing the foam is to employ the use of a foam in place process. Various areas of a trim panels of a vehicle, such as the cluster brow of an instrument panel, have relatively small height to depth (H/D) ratios. For portions of trim panels having a sprayed skin and height to depth ratios (H/D) of less than 1, the typical foam in place process may have difficulty being employed. This is because due to the low height to depth (H/D) ratio, line of sight to the entire portion of the cluster brow may be obstructed. As such, it can be difficult to spray skins of high quality. A relatively common result is that, due to obstructed line of sight, the spraying of the polyurethane skin may not result in complete coverage. This can result in the foam showing through to a vehicle occupant.

One technique for overcoming this drawback is to over spray the polyurethane skin these areas where line of sight is obstructed. It is believed that over spraying, while not necessarily being a cost effective use of materials, will result in complete coverage of the skin so that the foam is prevented from showing through. One drawback that has been associated with this method is that, due to the relatively higher density of the sprayed in skin relative to the foam, the resulting over sprayed area can become relatively heavy, dense, and hard. In addition to an undesirable hard feel, this can cause these areas, such as the cluster brow, to sag relative to the rest of the instrument panel.

### SUMMARY OF THE INVENTION

Under the invention, a method in making a vehicle interior trim panel is provided. The method includes providing a spray mold having a mold surface with a cavity having at least a portion having a height to depth (H/D) ratio of less than 1. The method further includes spraying polyurethane material proximate the surface of the tool to form a polyurethane skin layer. The method further includes spraying expandable polyurethane material onto the skin layer to form a resilient layer. The method further includes removing the skin layer and the resilient layer from the tool, positioning the skin layer and the resilient layer in a mold in a spaced apart relationship from a substrate, and introducing a foam material between the substrate and the resilient layer to form a foam layer between the substrate and the resilient layer.

In at least another embodiment, the resilient layer has a portion having a height to depth ratio of less than 1. In at least another embodiment, the resilient layer has a portion having a height to depth ratio of 0.1 to 0.9. In at least another embodiment, the resilient layer has a portion having a height to depth ratio of 0.1 to 0.5. In at least one embodiment, the skin layer is substantially the same color as the resilient layer.

While exemplary embodiments in accordance with the invention are illustrated and disclosed, such disclosure should not be construed to limit the claims. It is anticipated that various modifications and alternative designs may be made without departing from the scope of the invention.

### BRIEF DESCRIPTION OF THE DRAWINGS

FIG. 1 is perspective view of an instrument panel according to the invention for use in motor vehicle;

FIG. 2 is a cross sectional view of the panel taken along the line 2-2 of FIG. 1;

FIG. 3 is a schematic view of a spray mold and a spray assembly for use in forming the panel, wherein the spray assembly is shown applying material on the tool to form a coating;

FIG. 4 is a schematic view of the tool showing application of additional material onto the coating to form a skin layer;

FIG. 5 is a schematic view of the tool showing application of expandable material onto the skin layer to form a resilient layer; and

FIG. 6 is a schematic view of the coating, skin, and resilient layers spaced from a substrate between first and second mold portions of a mold and showing application of a foam material between the resilient layer and the substrate.

### DETAILED DESCRIPTION OF THE PREFERRED EMBODIMENT(S)

As required, detailed embodiments of the present invention are disclosed herein. However, it is to be understood that the disclosed embodiments are merely exemplary of the invention that may be embodied in various alternative forms. The figures are not necessarily of scale, some features may be exaggerated or minimized to show details of particular components. Therefore specific structural and functional details disclosed herein are not to be interpreted as limiting, but merely as a representative basis for the claims and/or a representative basis for teaching one skilled in the art to variously employ the present invention. Moreover, except where otherwise expressly indicated, all numerical quantities in this description and in the claims indicating amounts of materials or conditions of reactions and/or use are to be understood as modified by the word “about” in describing the broader scope of this invention. Practice within the numerical limits stated is generally preferred. Also, unless expressly stated to the contrary, percent “parts of,” and ratio values are by weight and the description of a group or class of materials as suitable preferred for a given purpose in connection with the invention implies that mixtures of any two or more members of the group or class may be equally suitable or preferred.

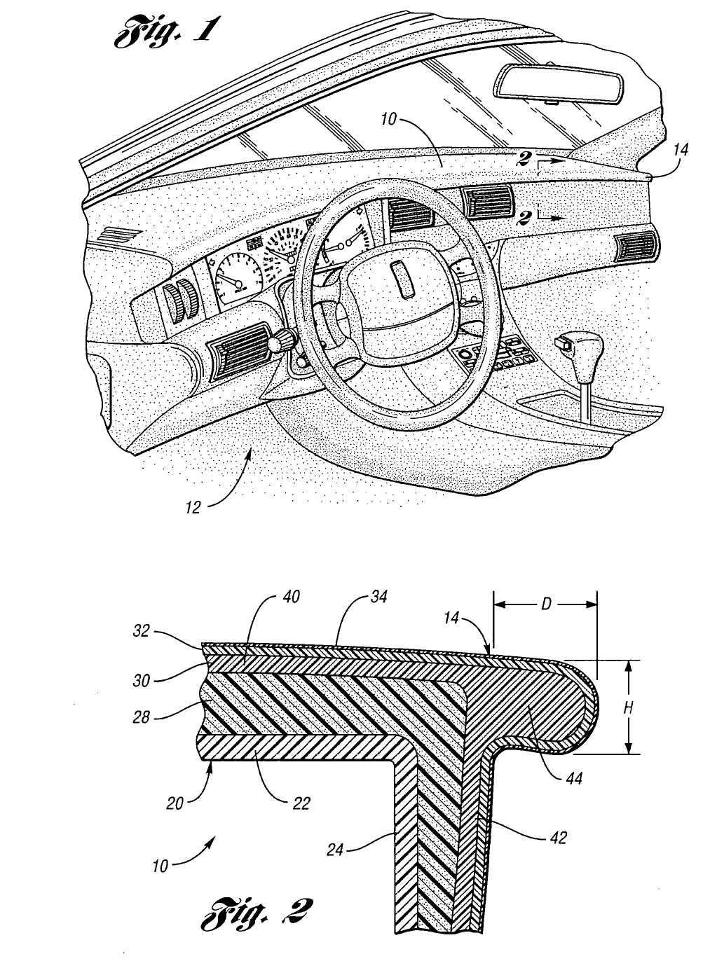

FIGS. 1 and 2 show an interior vehicle trim component, such as an instrument panel 10, according to the invention for use with a motor vehicle 12. In at least one embodiment, the instrument panel 10 has a brow portion 14 that has at least a portion having a height to depth ratio (H/D) of less than 1, in at least one embodiment between 0.1 and 0.9, and in yet at least another embodiment between 0.1 to 0.5.

In at least one embodiment, the panel 10 includes a rigid substrate 20 having a first portion 22, extending in a first, generally horizontal, direction, and a second portion 24 extending generally transverse to the first portion. The panel 10 further includes, in at least one embodiment, a foam layer 28 secured to the substrate 20, a resilient layer 30 secured to the foam layer 28, a skin layer 32 secured to the resilient layer 30, and a coating 34 that covers the skin layer 32 and provides an exterior apparent surface.

The substrate 20 is a structural member that provides support for the remainder of the panel 10, and may comprise any suitable material. For example, the substrate 20 may be made of plastic or reinforced plastic such as fiberglass reinforced polyurethane. Additional examples of suitable plastics, besides polyurethane, include polypropylene, polyethylene, acrylonitrile butadiene styrene (ABS), polycarbonate (PC), and ABS/PC blends. In at least one embodiment, the substrate may have a general thickness of between 0.5 to 5 mm, in another embodiment 1.0 to 3.5 mm, and in yet another embodiment 2.0 to 3.0 mm. While the portions 22 and 24 are shown to extend generally transverse to each other, it should be understood that they can extend in a variety of directions, besides transverse, and angles from each other.

The foam layer 28 is adhered between the substrate 20 and the resilient layer 30. The foam layer 28 generally contours to the substrate 20. The foam layer 28 helps to provide a soft feel to the instrument panel 10. The foam layer 28 may comprise any suitable foam material. For example, the foam layer 28 may comprise a foam in place polyurethane foam. In at least one embodiment, the density of the foam layer 28 is in the range of 0.05 to 0.20 grams per cubic centimeter (g/cm³). In at least one embodiment the foam layer 28 has a thickness of 3.0 to 25 mm, and in at least another embodiment 5 to 15 mm, and in yet another embodiment 8 to 12 mm. The foam layer 28 may have varying thicknesses. For instance, as shown in FIG. 2, the foam layer portion above the first portion 22 of substrate 20 may be generally thicker than the portion adjacent and abutting second portion 24 of the substrate 20.

The resilient layer 30 is adhered to the foam layer 28 and the skin layer 32. The resilient layer 30 may help to provide a soft feel to the panel 10. The resilient layer 30 may comprise any suitable sprayed expanded polyurethane material. For example, the resilient layer 30 may comprise an expanded aromatic polyurethane elastomer. In at least one embodiment, the density of the resilient layer 30 can be in the range of 0.1 to 0.75 g/cm³, in another embodiment between 0.15 to 0.5 g/cm³, and in yet another embodiment 0.2 to 0.3 g/cm³. In at least in one embodiment, the resilient layer 30 may have a color that would be aesthetically pleasing to a vehicle customer. In at least one embodiment, the resilient layer 30 may have a shore A hardness of 30 to 50.

In at least one embodiment, the resilient layer 30 primarily may comprise a low permeable, relatively closed cell material. In at least one embodiment, the resilient layer 30 may comprise 35 to 75 percent closed cell structures, in at least another embodiment 40 to 65 percent closed cell structures, and in yet at least another embodiment 45 to 55 percent closed cell structures, based upon the entire resilient layer 30.

In at least one embodiment, the resilient layer 30 may have varying closed cell density throughout the resilient layer. In at least one embodiment, the top portion (the portion closer to the skin layer 32) and the lower portion (the portion closer to the foam layer 28) may independently comprise at least 85 percent closed cell structures, in other embodiments between 90 to 100 percent closed cell structures, and in yet other embodiments between 95 and 100 percent closed cell structures. In at least one embodiment, the top and bottom portions may independently comprise between 2.5 to 25 percent, in other embodiments between 5 to 20 percent, and in yet other embodiments between 10 to 15 percent, of the thickness of the resilient layer 30. The remaining intermediate layer (the portion between the top and bottom portion) of the resilient layer 30 may comprise between 40 to 95 percent closed cell structures, in at least another embodiment between 50 to 90 percent closed cell structures, and in yet another embodiment between 60 to 85 percent closed cell structures. In at least one embodiment, the average cell structure size can vary between 0.05 mm to 3.0 mm, and in yet other embodiment between 0.5 mm to 1.0 mm.

The resilient layer 30 generally comprises a first portion 40 extending generally in a horizontal direction, a second portion 42 extended in a second direction, generally transverse to the first direction, and a nose portion 44 extending from and between the first and second portions 40 and 42, respectively. The first and second portions 40 and 42, respectively, generally contour to the foam layer 28. In at least one embodiment, the nose portion 44 has at least a portion, in other embodiments a substantial portion, and in yet other embodiments, all of it, having a height to depth (H/D) ratio of less than 1.0 in at least one embodiment, in at least another embodiment of 0.1 to 0.9, and in yet at least another embodiment of 0.1 to 0.5. The nose portion 44 gives the general shape to the brow portion 14 of the panel 10. In at least one embodiment, the first and second portions 40 and 42 respectively, of the resilient layer 30 may have thicknesses generally between 0.1 to 30 mm, in other embodiments between 1 to 15 mm and in yet other embodiments 2 to 10 mm. The thicknesses of the portions 40 and 42 can vary from each other, as is shown in FIG. 2.

The skin layer 32 is adhered to the resilient layer 30 and coating 34. Moreover, the skin layer 32 is configured to provide a covering over, and is generally contoured to, the resilient layer 30 and may comprise any sufficiently dense material. For example, the skin layer 32 may be a solid layer that comprises an aromatic or aliphatic compound. As a more specific example, the skin layer 32 may be made of an elastomer such as polyurethane. Furthermore, the skin layer 32 may have any suitable thickness and density. For example, the skin layer 32 may have a thickness in the range of 0.4 to 2 mm and a density in the range of 0.85 to 1.2 g/cm³. In at least one embodiment, the skin layer 32 has a thickness in the range of 0.5 to 1.2 mm, and a density in the range of 0.95 to 1.1 g/cm³. In at least one embodiment, the skin layer 32 has a color that is substantially the same, or is the same, as the color of the resilient layer 30. This helps to prevent uneven coloration of the panel 10 if the skin layer 32 coverage is less than complete.

The optional coating 34 may be used to protect the skin layer 32 and/or to provide a decorative surface for the instrument panel 10. For example, the coating 34 may be used to inhibit sunlight and/or other ultraviolet light from reaching the skin layer 32. As another example, the coating 34 may be used as a paint to provide a desired color and/or texture to the instrument panel 10. While the coating 34 may comprise any suitable material, in at least one embodiment of the invention, the coating 34 is made of an aliphatic polyurethane composition. Furthermore, the coating 34 may have any suitable thickness, such as a thickness of approximately 0.5 to 1 mil.

Alternatively, the coating 34 may be omitted if not required for a particular application. For example, the skin layer 32 may be configured to provide a sufficiently durable and attractive surface such that the coating 32 is not needed. As such, if the coating 34 is not present either in whole or in part, the skin layer 32, and in areas of insufficient skin layer coverage, the resilient layer 30, may provide a desired color and/or texture to the instrument panel 10.

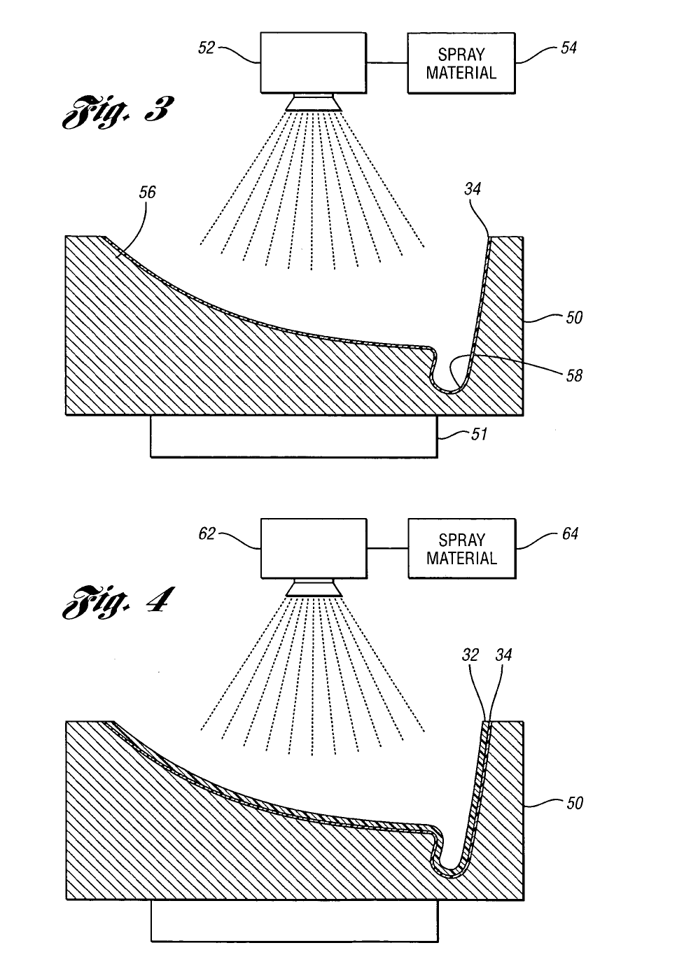

Referring to FIGS. 3-6, a method of manufacturing the instrument panel 10 will now be described. The method may begin by spraying an optional mold release agent and then the optional coating 34 on a spraying mold tool 50 using any suitable device, such as robotic low pressure (such as 10 to 40 psi) spray assembly 52 having one or more moveable spray nozzles. A source 54 of coating forming material is in fluid communication with the spray assembly 52.

The tool 50 is supported on base 51 and has a spray receiving surface 56 generally corresponding to the surface of the panel 10. The spray surface 56 has a cavity 58 generally corresponding to the surface of the brow portion 14 (and nose portion 44) of the panel 10. As such, in at least one embodiment, the cavity 58 has at least a portion having a height to depth ratio (H/D) of less than 1, and in at least another embodiment between 0.1 and 0.9, and in yet at least another embodiment between 0.1 to 0.5. As can be understood, a substantial portion or even all of the cavity 58 could have a height to depth ratio (H/D) of less than 1, and in at least another embodiment between 0.1 and 0.9, and in yet at least another embodiment between 0.1 to 0.5. The tool 50 may be heated to any suitable temperature if desired.

Next, referring to FIG. 4, the method involves spraying skin layer forming material onto the coating 34 to form skin layer 32, which bonds to the coating 34. A source of skin layer forming material 64 is in fluid communication with the spray assembly 62. The skin layer forming material may be any suitable sprayable substance and may be applied using any suitable device. For example, the material may include polyol and isocyanate, and the material may be sprayed with a robotic high pressure (such as 400 to 2,000 psi) spray assembly 62 having one or more moveable spray nozzles. As a result, the skin layer 32 may be formed of polyurethane.

As mentioned above, the coating 34 may be omitted from the panel 10. In such a case, the skin forming material may be sprayed directly onto the tool 50, or onto a mold release agent that is applied to the tool 50, to form the skin layer 32. In this embodiment, the skin layer 32 is preferably a colored aliphatic polyurethane.

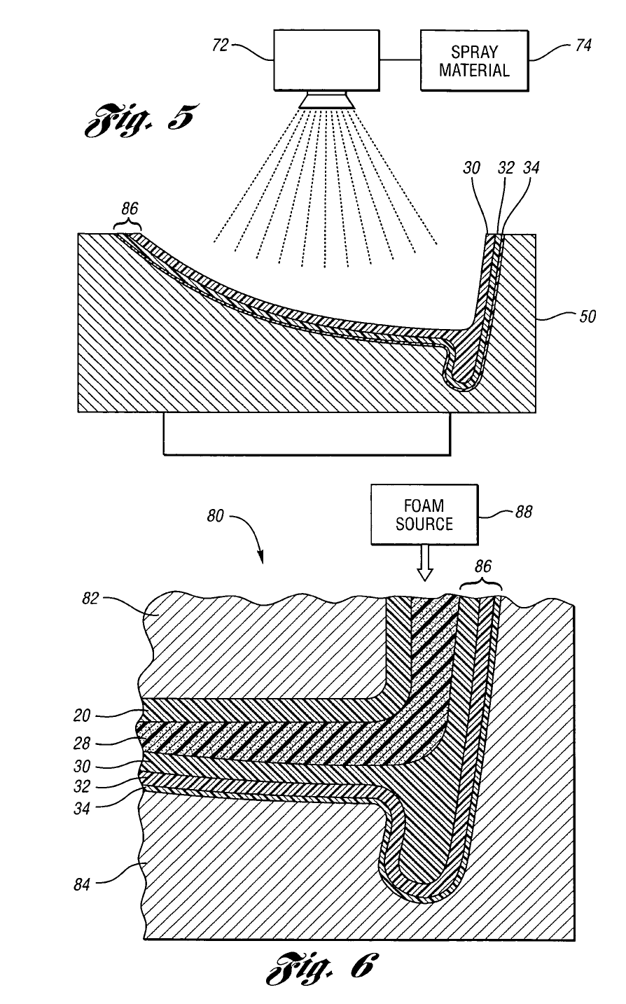

Referring to FIG. 5, the method then involves introducing expandable material onto the skin layer 32 to form resilient layer 30, which bonds to the skin layer 32. The expandable material may comprise any suitable expandable material. In at least one embodiment, the expandable material comprises any suitable expandable polyurethane material and may applied in any suitable manner. For example, the expandable polyurethane material may comprise polyol, isocyanate and a blowing agent such as water and/or a readily volatile organic substance, such as a delayed-action amine catalyst. In certain embodiments, specific examples of suitable catalysts are the delayed-action amine catalyst DABCO® BL-17 and the amine catalyst DABCO® BL-22 available from Air Products and Chemicals, Inc. of Allentown, Pa.

Furthermore, the expandable material may be sprayed with a robotic spray assembly 72 which receive the expandable material from a source 74 of expandable material in fluid communication with the spray assembly 72. The robotic spray assembly 72 may include one or more spray nozzles and the material may be allowed to free rise to achieve a desired density. The robotic spray assembly 72 may be the same type of spray assembly 62 as is used in FIG. 4. Alternatively, the same spray assembly 62 as used in FIG. 4 could be used to spray the expandable material. The expandable material, in at least one embodiment, can be a similar color to the color of the skin material 32 such that the skin material 32 and the resilient layer 30 are generally the same color. Furthermore, in at least one embodiment, the expandable material is generally sprayed longer in the area of the cavity 58 to achieve a desired thickness and to fill in the cavity 58 such that the nose portion 44 is formed between the first and second portions 40 and 42, respectively, of the resilient layer 30. In at least one embodiment, nose portion 44 can generally be 1 to 25, and in other embodiments 5 to 20, times the thickness, or depth D, than either of the first or second portions 40 and 42, respectively.

Next, referring to FIG. 6, the method involves removing the optional coating 34, the skin layer 32 and the resilient layer 30, which comprises composite skin 86, from the mold tool 50 and positioning the composite skin 86 in a foam in place mold 80 having a first mold portion 82 and a second mold portion 84. At least one of the mold portions 82 and 84 are movable relative to the other. In particular, the composite skin 86 comprising the coating 34, the skin material 32 and the resilient layer 30, in at least one embodiment, is provided on the second mold portion 84.

A substrate 20 may be suitably provided on the first mold portion 82 in a spaced apart relation from the composite skin 86. The mold portions 82 and 84 may then be closed together, and foam material can be injected at a relatively low pressure (such as 15 to 30 psi), from foam source 88, into the mold 80 through one or more injection passages (not shown) to form the foam layer 28, which bonds to the substrate 20 and the resilient layer 30. The instrument panel 10 may then be removed from the mold 80.

Examples of other vehicle parts that may be manufactured by the above method includes door panels, package shelves, pillar trim panels, trim products, door covers, console covers, shelves, and trim covers, among others.

Although separate spray assemblies 62 and 72 are shown in the figures, the spray assemblies may be provided as a single spray assembly. Furthermore if the material to be sprayed includes multiple substances, the substance may be mixed at a suitable time. For example, the substances may be mixed up stream of, within, or down stream of the associated spray nozzles.

While embodiments of the invention have been illustrated and described, it is not intended that these embodiments illustrate and describe all possible forms of the invention. Rather, the words used in the specification are words of description rather than limitation, and it is understood that various changes may be made without departing from the spirit and scope of the invention.

What is claimed is:

1. A method for making a vehicle interior trim panel, said method comprising:
providing a spray mold having a mold surface having a cavity having at least a portion having a height to depth (H/D) ratio of less than 1.0,
spraying polyurethane material proximate the surface of the tool to form a polyurethane skin layer;
spraying expandable polyurethane material onto the skin layer to form a resilient layer;
removing the skin layer and the resilient layer from the tool and positioning the skin layer and the resilient layer in a mold in a spaced apart relationship from a substrate; and
introducing a foam material between the substrate and the resilient layer to form a foam layer between the substrate and the resilient layer.

2. The method of claim 1 wherein the skin layer has a predetermined first color and the resilient layer has a predetermined second color substantially similar to the first color.

3. The method of claim 2 wherein the second color is the same as the first color.

4. The method of claim 1 wherein the cavity has a portion having a height to depth (H/D) ratio of 0.1 to 0.5.

5. The method of claim 4 wherein the resilient layer has a first portion extended generally in a first direction, a second portion extending generally in a second direction, generally transverse from the first direction, and a nose portion extending between and connecting the first and second portions, the nose portion having a height to depth ratio of less than 1.0.

6. The method of the claim 1 wherein the nose portion has a substantial portion having a height to depth ratio of 0.1 to 0.5.

7. The method of claim 6 wherein the resilient layer has a density of 0.1 to 0.75 g/cm³.

8. The method of claim 7 wherein the skin layer has a density of 0.85 to 1.2 g/cm³.

9. The method of claim 8 wherein the first and second portions of the resilient layer have a thickness of 0.1 to 6 mm.

10. A vehicle interior trim component for use with a motor vehicle, the trim component comprising:
a sprayed polyurethane skin layer;
a sprayed expanded polyurethane resilient layer secured to the skin layer, the resilient layer having a first portion extending in a first direction, a second portion extending in a second direction, generally transverse to the first direction, and a nose portion extended between and connecting the first and second portions, the nose portion having at least a portion having a height to depth ratio of less than 1.0; and
a rigid substrate secured to the resilient layer.

11. The vehicle interior trim component of claim 10 wherein the skin layer has a predetermined first color and the resilient layer has a predetermined second color substantially similar to the first color.

12. The vehicle interior trim component of claim 11 wherein the second color is the same as the first color.

13. The vehicle interior trim component of claim 10 wherein the nose portion has a height to depth (H/D) ratio of 0.1 to 0.5.

14. The vehicle interior trim component of claim 10 wherein the nose portion has at least a substantial portion having a height to depth ratio of 0.1 to 0.5.

15. The vehicle interior trim component of claim 10 wherein the resilient layer has a density of 0.1 to 0.75 g/cm³.

16. The vehicle interior trim component of claim 15 wherein the skin layer has a density of 0.85 to 1.2 g/cm³.

17. The vehicle interior trim component of claim 16 wherein the first and second portions of the resilient layer have a thickness of 0.1 to 6 mm.

18. A method for making a vehicle interior trim panel, said method comprising:
providing a spray mold having a mold surface having a cavity having a portion having a height to depth (H/D) ratio of less than 1.0,
spraying polyurethane material proximate the surface of the tool to form a polyurethane skin layer;
spraying expandable polyurethane material onto the skin layer to form a resilient layer, the resilient layer having a first portion extended generally in a first direction, a second portion extending generally in a second direction, generally transverse from the first direction, and a nose portion extending between and connecting the first and second portions, the nose portion having a height to depth ratio of less than 1.0, the expanded polyurethane material being sprayed longer in the vicinity of the cavity than in the other potions of the mold surface;
removing the skin layer and the resilient layer from the tool and positioning the skin layer and the resilient layer in a mold in a spaced apart relationship from a substrate; and
introducing a foam material between the substrate and the resilient layer to form a foam layer between the substrate and the resilient layer.

19. The method of claim 18 wherein the skin layer has a predetermined first color and the resilient layer has a predetermined second color substantially similar to the first color.

20. The method of claim 19 wherein the resilient layer has a density of 0.1 to 0.75 g/cm³.
